(19) United States
(12) Patent Application Publication
(10) Pub. No.: US 2013/0184588 A1
(43) Pub. Date: Jul. 18, 2013

(54) DETECTING A STENOSIS IN A BLOOD VESSEL

(71) Applicant: Echosense Inc., (US)

(72) Inventor: Yoram Palti, Haifa (IL)

(73) Assignee: ECHOSENSE INC., TORTOLA (VI)

(21) Appl. No.: 13/787,345

(22) Filed: Mar. 6, 2013

Related U.S. Application Data
(62) Division of application No. 12/700,828, filed on Feb. 5, 2010.
(60) Provisional application No. 61/150,146, filed on Feb. 5, 2009.

(51) Int. Cl. A61B 8/06; A61B 8/08

(52) U.S. Cl. CPC A61B 8/06; A61B 8/488; A61B 8/5223
USPC 600/454

(57) ABSTRACT

Doppler ultrasound may be used to detect stenosis in a blood vessel using a variety of approaches. In one approach, the flow envelope is extracted from the Doppler ultrasound measurements, and the extracted flow envelope is parameterized. Classification is then done based on those parameters (and optionally other parameters), to determine whether a stenosis exists. A second approach uses Doppler data that is acquired in a direction that is perpendicular to the direction of blood flow, and detects artifacts that are consistent with turbulences that usually appear downstream from stenoses.

### CROSS REFERENCE TO RELATED APPLICATIONS

This application is a divisional of U.S. application Ser. No. 12/700,828, filed Feb. 5, 2010, which claims the benefit of U.S. provisional application 61/150,146, filed Feb. 5, 2009, each of which is incorporated herein by reference.

### BACKGROUND

In general, the flow velocity in a stenosed artery increases in direct proportion to the degree of stenosis (i.e., the relative reduction in cross section area of the vessel). However, under certain conditions, the general rule breaks down.

The flow (Q) in a normal artery segment is dependent on the pressure drop (ΔP) along the vessel and on the overall resistance (R) to flow, which normally resides in the intra-myocardium vessels. In case of a stenotic section, a local resistance to flow, that is determined by the restriction dimensions, is added to the peripheral resistance.

Q=ΔP/R=ΔP/(Rstenosis+Rmyocard) [cm³/min]

The resistance to flow at the stenosed section depends on the blood viscosity (μ), the length of the stenosis (L) and the radius (r) of the stenosed vessel such that:

Q=ΔP/(8 μL/π⁴ +Rmyocard)

The flow velocity (V) in the stenotic section is inversely proportional to the average cross section area, relative to the normal artery cross section, which defines the degree of stenosis (assuming that the flow remains constant).

V=Q/π² [cm/sec]

The variation of the flow and the velocity in the stenotic section, as a function of the degree of the stenosis, are shown in FIG. 1 which is a simulation of coronary artery flow rate and flow velocity as a function of the level of stenosis in a 1 cm long segment. The calculation is made using the following parameters:
Rmyocard=60 mm Hg/cm³/sec
μ blood=0.045*P (gr/cm*sec).
ΔP along the blood vessel=70 mm Hg
Normal coronary Radius=1.5 mm
L stenotic length=10 mm

In FIG. 1, curve 12 is the flow rate in the stenosed segment, which is almost constant up to 50% stenosis and then drops to half the initial value at about 75% stenosis. This reduction in flow rate eventually results in an attenuation of flow velocity (curve 14) in the stenosed segment. Thus the velocity reaches a maximum at about 75% stenosis and then declines steeply towards zero. The fact that the flow velocity in a highly stenosed artery may be lower than in a mildly stenosed one was demonstrated experimentally in the lab and clinically. Because of this, it is not possible to use blood flow velocity measurements alone (e.g., as determined over the chest wall using a Doppler system) to determine the degree of arterial stenosis. Note that when a severe stenosis is present, the reduction in flow rate also results in a reduction of the flow velocity (curve 16) in the non-stenosed segment.

One aspect of the invention relates to a method of detecting a flow disturbance in a vessel through which a fluid is flowing. This method includes the steps of obtaining Doppler ultrasound measurements of fluid flow through the vessel, extracting a flow envelope from the Doppler ultrasound measurements, parameterizing the flow envelope to generate a first set of parameters, and performing classification to determine whether a flow disturbance exists in the vessel based on the first set of parameters.

Another aspect of the invention relates to a method of detecting a stenosis in a coronary blood vessel. This method includes the steps of obtaining Doppler ultrasound measurements of blood flowing through the vessel, extracting a flow envelope from the Doppler ultrasound measurements, parameterizing the flow envelope to generate a first set of parameters, and performing classification to determine whether a stenosis exists in the vessel based on the first set of parameters. The first set of parameters includes at least (a) a parameter for the largest difference in maximum power between adjacent intercostal spaces, (b) a parameter for Mean Power for all velocities in a period, and (c) a parameter for peak velocity time interval.

Another aspect of the invention relates to a method of detecting a stenosis in a vessel through which a fluid is flowing. This method includes the steps of generating a beam of ultrasound energy, aiming the beam at a point in the vessel at an angle of less than 20° with respect to a plane that (a) is perpendicular to the direction of flow in the vessel and (b) passes through the point, using Doppler processing to detect (within the vessel) velocity components of fluid motion that are perpendicular to the direction of fluid flow, repeating the aiming step and the using Doppler processing step at a plurality of points in the vessel, identifying a location in the vessel at which the detected velocity components have high power at high velocities, and determining that there is a high likelihood that a stenosis is present at a position that is upstream from the identified location.

Yet another aspect of the invention relates to a method of detecting a stenosis in a vessel through which a fluid is flowing. This method includes the steps of generating a beam of ultrasound energy, aiming the beam at a point in the vessel at an angle of less than 20° with respect to a plane that (a) is perpendicular to the direction of flow in the vessel and (b) passes through the point, using Doppler processing to detect (within the vessel) velocity components of fluid motion that are perpendicular to the direction of fluid flow, and displaying an indication of a power level for the detected velocity components. In instances where a high power level for high velocity components is present, the presence of the high power level for high velocity components is correlated with the presence of a stenosis in the vessel.

### BRIEF DESCRIPTION OF THE DRAWINGS

FIG. 1 is a graph that describes flow characteristics in a stenosed segment.

FIG. 2 is a flowchart of one approach for implementing a multi-parameter analysis to detect stenoses or other abnormal flows in an artery or other vessel.

FIG. 3 is a (velocity and power) vs. time plot for flow in an artery.

FIG. 4 is a plot depicting a flow envelope.

FIGS. 5A and 5B are schematic representations of flow in a vessel with a stenosis, in side and cross section views, respectively.

FIG. 6A is a (velocity and power) vs. distance plot for a stenosed artery.

FIG. 6B is a power vs. distance plot for a stenosed artery of FIG. 6A.

FIG. 7A is a set of Power Spectra for various flow rates and stenosis levels.

FIG. 7B shows the correlation between the positive and negative in FIG. 7A.

FIGS. 8A, 8B, and 8C are power spectra for three different scenarios of blood flow in a vessel.

FIG. 9 is a flowchart depicting how the Multi-Parameter approach for detecting a stenosis can be combined with the Perpendicular Data approach for detecting a stenosis

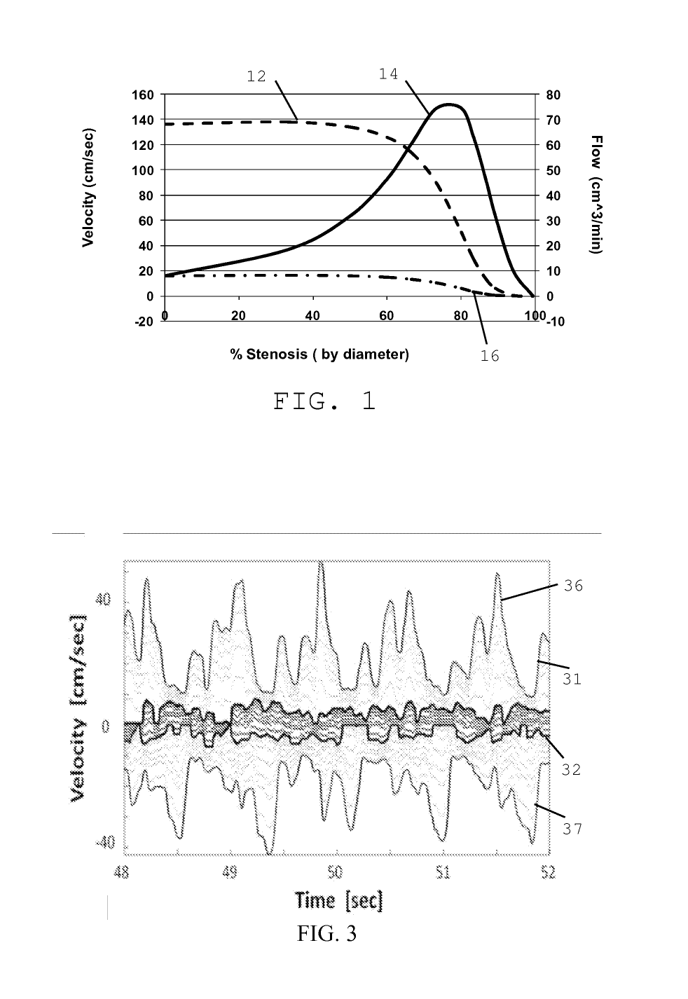

### DESCRIPTION OF THE PREFERRED EMBODIMENTS

Two approaches are described herein for overcoming the above problem, and for diagnosing and characterizing stenoses based on Doppler measurements. The first approach uses a multi-parameter analysis of Doppler data. The second approach uses Doppler data that is acquired in a direction that is perpendicular to the direction of blood flow, a direction that was traditionally thought to be useless for this purpose. Optionally, these two approaches can be combined.

I. Multi-Parameter Analysis of Doppler Data

The first approach uses parametric characterization of fluid flow in vessels, including flow under varying pressure and flow in vessels the cross section of which is not constant, i.e. they have one or more narrowing, such as stenoses in blood vessels, or a widening (aneurisms), etc. Characterization of the flow rate, velocity, power, time course, and duration of the parameters, and combinations of all of the above, are made. The data analyses can be made on-line or off-line.

The following description will relate, as an example, to flow of blood in blood vessels in general and the coronary arteries in particular, and to phantoms of such systems as measured by Doppler ultrasound. The prime targets of the flow parameterization and characterization are to detect and diagnose stenoses in arteries or other vessels, the presence of changes in vessel walls and diameter, as well as to determine the functional state of the vessel and the fluid flow through it. The parametric characterization spans the whole spectrum of flow disturbances, from relatively small narrowing/widening and vessel lining defects, including those defined as vulnerable plaques, through sever narrowing/widening (stenoses & aneurisms) and up to complete vessel occlusion. Note that while the embodiments set forth herein are described primarily in the context of stenoses in coronary arteries, the techniques described herein are not limited to that particular context, and may also be used to detect other types of flow disturbances in coronary arteries or other blood vessels. They may also be used to detect stenoses and other flow disturbances in other types of fluid circuits (e.g., in biological and industrial applications).

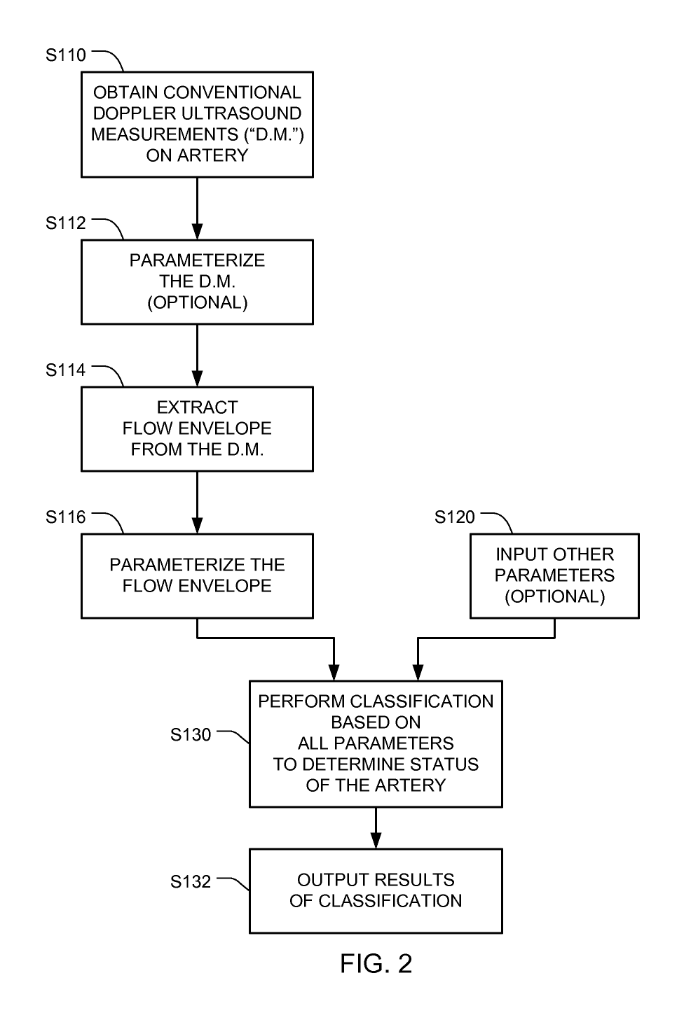

FIG. 2 is a flowchart of one approach for implementing a multi-parameter analysis to detect stenoses or other abnormal flows in an artery or other vessel. In step S110, Doppler ultrasound measurements of the relevant artery are obtained using any conventional approach. Preferably, these ultrasound measurements are parameterized in step S112. Examples of parameters that can be obtained from the conventional ultrasound measurements are included in Tables 1 and 2, below.

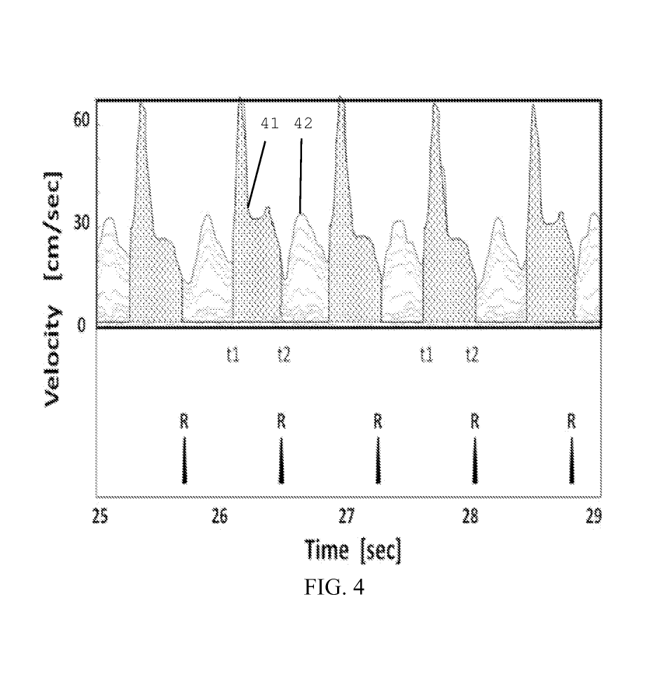

In step S114, the flow envelope is extracted from the ultrasound measurements. One suitable way to accomplish this step is to start with conventional (velocity and power) vs. time data. An example of this data is depicted in FIG. 3. Conventionally, this type of data is displayed with power denoted by color. But in FIG. 3, the color has been replaced grayscale. Starting with this power-velocity signal tracing vs. time data, pre-processing algorithms are preferably applied to (a) separate the fluid velocity from the wall motion, and (b) separate the fluid velocity from the noise.

In FIG. 3, the contour plots show the maximal velocities picked up by Doppler signals originating either from cardiac muscle movement or coronary flows during transthoracic coronary artery Doppler examination. More specifically, FIG. 3 shows the contours of the maximal values of the velocity of both the cardiac wall motion (traces 31, 32, which are closest to the zero line) and the maximal blood flow velocity (traces 36, 37, which are the upper most and lower most traces).

A suitable pre-processing algorithm for distinguishing between blood flow in vessels and non-specific noise may be implemented using the following two stage process. (Stage 1) Define, at any given time (tᵢ), a threshold ‘thr(t₁)’ for each power spectrum A(tᵢ) as follows: Search for a region of lowest energy in the proximity of tᵢ. thr(tᵢ) is equal to the highest power level in this region. Then apply thr(ti) on A(ti)—all parts of A(tᵢ) above thr(tᵢ) are flow regions and other parts are noise. (Stage 2) Refine of the initial distinction between flow and noise by using the statistics of noise. Assume down estimation (flow being included in noise region). Adjust envelopes detection to exclude flow pixels from noise regions. Identify pixels of flow in noise regions by their relatively high values.

A suitable pre-processing algorithm for distinguishing between blood flow in vessel and tissue motion (cardiac wall motion) may be implemented as follows. Note that this algorithm is preferably applied after the noise removing algorithm described above or another suitable noise removing algorithm. Accordingly, at this point we assume that the data includes two sub-regions—blood flow and tissue motion, defined as ROI1. The algorithm includes the following steps:
(1) Divide ROI1 along time to sub regions ROI2ⱼ, such that ∪ {ROI2ⱼ}=ROI1. For example—define ROI2ⱼ as an interval of 4 heart beats.
(2) For each j=1,2, . . . , J, Detect locations {t,v} and power levels {p} of local peaks of power level of the spectrogram (bright spots) in ROI2ⱼ.
(3) Define a threshold which satisfies the condition: P(p<thrⱼ)=pₜₕᵣ.
(4) Start with pₜₕᵣ=0.7, change the initial value to improve edge detection.
(5) Use thrⱼ to divide the bright spots to two groups—each spot of p<thrj is related to the region of blood flow, and is marked as {t^{bf},v^{bf}}. All other points are related to the region of tissue motion, and are marked as {t^{tm},v^{tm}}.
(6) For each point (ti,vi) within ROI2j calculate two distances:

d^{bf}=d({t^{bf},v^{bf}},(ti,vi)), and d^{tm}=d{t^{tm},v^{tm}},(ti,vi))
(7) If d^{bf}<d^{tm} relate (ti,vi) to blood flow region. Otherwise, relate (ti,vi) to tissue motion region.
(8) Reject outliers and define a clear cut edge (as a function of time) between blood flow and tissue region.

Another pre-processing algorithm that may be applied at this point is the Reduction of Tissue Motion effect on blood flow Power Levels Distribution, to balance power distribution in tissue motion according to power distribution in blood flow regions. One suitable approach for implementing this is as follows: For each time t, Shift the local histogram of power levels of tissue motion region towards the local histogram of power levels of blood flow region, to achieve equal average values of the two distributions.

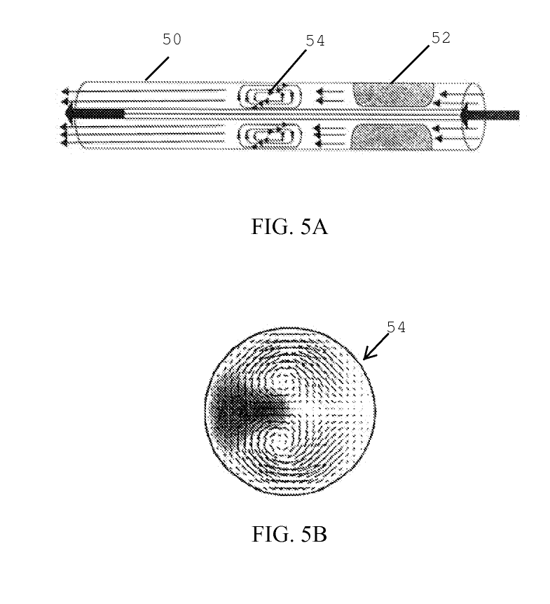

After these pre-processing algorithms for edges detection and tissue reduction are applied, we obtain the flow envelope data depicted in FIG. 4, in which the regions of blood flow (for example diastolic flow 41) are defined by the t1 & t2 intervals, and R indicates the R wave of the ECG. Returning to FIG. 2, this concludes step S 114.

After the flow envelope has been extracted, it is parameterized in step S116. The following is a partial list of the parameters that may be used to characterize the flows so as to diagnose and estimate the extent of various defects in the arteries, or other vessels. Some of the data is derived from the power spectra themselves as provided by the Doppler measurements. The features of these power spectra may also be parameterized, for example the power at specific velocities, the average slopes of the curves, the number of different slopes at the positive and negative sides of the spectra, etc. Parameters may also be derived from the velocity and power versus time tracings. Note that parameters may be derived separately from the diastolic portion of flow envelope (41 in FIG. 4) or from the systolic portion of flow envelope (42 in FIG. 4), or both of those portions taken together. Table 1 lists some examples of the above for scalar velocity features, and Table 2 lists some examples of the above for scalar power features.

TABLE 1
Scalar Velocity Features

$VTI = Δt \cdot {\sum }_{t = t 1}^{t 2} {flow\_envelope}_{( t )}$ $ADPV = \frac{1}{t 2 - t 1 + 1} {\sum }_{t = t 1}^{t 2} {flow\_envelope}_{( t )}$
peak_velocity = max{flow_envelope}
$max\_slope = max \{ \frac{d}{dt} ( flow\_envelope ) \}$ $Mean\_weighted \_V = \frac{{\sum }_{t = t 1}^{t 2} {\sum }_{v = 0}^{{flow \_ envelope}_{( t )}} ( {P}_{( t , v )} \cdot v )}{{\sum }_{t = t 1}^{t 2} {\sum }_{v = 0}^{{flow \_ envelope}_{( t )}} {P}_{( t , v )}}$ $MMWVC = \frac{Δt \cdot {\sum }_{t = t 1}^{t 2} ( \frac{{\sum }_{v = 0}^{{flow \_ envelope}_{( t )}} ( {P}_{( t , v )} \cdot v )}{{\sum }_{v = 0}^{{flow \_ envelope}_{( t )}} {P}_{( t , v )}} )}{t 2 - t 1 + 1}$

TABLE 2
Scalar Power Features

Mean_power = mean{P_{(t, v)}}_{(t, v)εROI}
Max_power = max{P_{(t, v)}}_{(t, v)εROI}
Median_power = median{P_{(t, v)}}_{(t, v)εROI}
std_power_flow = std{P_{(t, v)}}_{(t, v)εROI}
std_power_flow_dB = std{10 · log₁₀(P_{(t, v)} + 1)}_{(t, v)εROI}
$PVTI = Δv \cdot Δt \cdot {\sum }_{t = t 1}^{t 2} {\sum }_{v = 0}^{{flow \_ envelope}_{( t )}} ( {P}_{( t , v )} \cdot v )$ $total\_power = Δv \cdot Δt \cdot {\sum }_{t = t 1}^{t 2} {\sum }_{v = 0}^{{flow \_ envelope}_{( t )}} {P}_{( t , v )}$

Optionally, in step 120, other parameters that are not derived from the Doppler data are obtained, using any conventional approach such as a keyboard or a touch screen user interface. Examples of such parameters are shown in Table 3.

TABLE 3
Other Features

Diastolic_flow_interval = t2 − t1
Age
Weight
Height

After the parameters are obtained as described above, additional parameters may be generated by performing various operations on the obtained parameters. Examples of suitable operations include: (a) calculating the Maximal value of each basic feature for each point of measurement (i.e., at each Inter-Costal Space—ICS₃, ICS₄, ICS₅, ICS₆); (b) calculating the differences-divided-by-averages between adjacent Inter-Costal Spaces, for example: (ICS₄−ICS₃)/(ICS₄+ICS₃); and (c) calculating the maximal difference for the purpose of per-patient analysis.

After all the relevant obtained and/or generated parameters are collected, classification is performed on those parameters to determine the status of the artery in step S130. The goal of the classification is to detection specific properties of clinical value (for example, determining whether a stenosis is present and the severity of any such stenosis).

This can be done, for example, as in the following two stage process:

### Stage 1—Learning:

A linear classifier is assumed to separate the data.
The classifier parameters are learned from the data using any suitable approach, based on a sample population of arteries that have stenoses of various severities and arteries with no stenoses. Classification may be done by a variety of approaches including but are not limited to LDA (Linear Discriminant Analysis) and SVM (Support Vector Machine) methods.
The resulting parameters are:
w—a vector of length N: w=[w₁,w₂, . . . , w_{N}]; and
b—a scalar
   Stage 2—classification:

Given a vector of features x=[x₁,x₂, . . . , x_{N}]
we use the classifier to calculate the linear combination:
f=sign(w₁*x₁+w₂*x₂+ . . . +w_{N}*x_{N}+b)
f can be equal to {−1,1}.
Depending on the outcome, (i.e., if f is −1 or +1), the subject is related to one group (e.g., the group in which a severe stenosis is present) or the other group (e.g., the group in which a severe stenosis is not present).

A classification system was implemented to determine whether a severe stenosis exists using the parameters and weights listed in Table 4, combined using the equation f=(w₁*x₁+w₂*x₂+ . . . +w_{N}*x_{N}). With those parameters and weights, a result of f that was below a threshold value of 0.2 indicated that a severe stenosis was present, and a result having f that was above 0.2 indicated the absence of a severe stenosis.

TABLE 4

| i | Parameter (xᵢ) | Weight (wᵢ) |
|---|---|---|
| 1 | Diastolic Flow Interval (time) | 0.39 |
| 2 | Mean Power (for all velocities in the period) | 1.01 |
| 3 | PVTI (peak velocity time interval) | −1.02 |
| 4 | Standard Deviation Power Flow dB | −0.76 |
| 5 | Diff max power | 1.11 |
| 6 | Diff VTI | 0.43 |
| 7 = N | Diff ADPV | 0.7 |

The first four of these parameters are self-explanatory. The equations for the final three parameters are as follows:

Diff_max_power=MAX{{max_power}ICS(i+1)−{max_power}ICS(i)}i=1 . . . n

Diff_VTI=MAX{{VTI}ICS(i+1)−{VTI}ICS(i)}i=1 . . . n

Diff_ADPV=MAX{{ADPV}ICS(i+1)−{ADPV}ICS(i)}i=1 . . . n

In all three of these equations, ICS(n) refers to the measurement made at the n^{th} intercostal space; VTI is the Velocity Time Integral, and ADPV is Average Diastolic Peak Velocity. Thus, the equation for Diff_max_power set forth above denotes calculating the difference in maximum power between adjacent intercostal spaces, and selecting the largest of all those differences (i.e., selecting the largest difference in maximum power between adjacent intercostal spaces).

Note that while Table 4 lists seven parameters that were determined to be important, alternative embodiment may use fewer or more parameters. For example, the top three or top four most highly weighted parameters in Table 4 may be used, taken alone or combined with other parameters, to perform the classification.

The results of the classification are then output in step S 132, using any conventional user interface.

II. Using Perpendicular Doppler Data

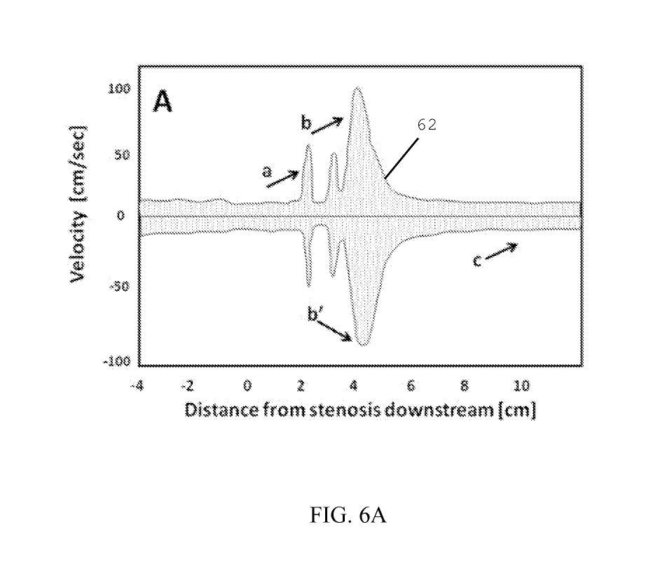

Normally, the flow in a tube or artery has no component in the plane normal to the flow axis therefore a probe positioned perpendicularly (at)90° or close to perpendicular to a blood vessel axis detects no Doppler signals, other than noise. However, as depicted in FIGS. 5A and 5B, it turns out that turbulence usually appears downstream from a stenotic segment. Turbulences include flow in multiple directions, i.e., directions other than flow along the axis of the vessel, including in the normal (90°) direction. FIG. 5A is a schematic presentation of a turbulence 54 that appears downstream from a stenosis 52 in a vessel 50, as seen in the side view of the flow along the vessel 50, and FIG. 5B is the flow pattern as seen in cross section at the same turbulence 54.

The inventors have recognized that useful information relating to stenoses can be obtained by examining such turbulences. One way to detect such turbulences is by using Doppler ultrasound flow measurement and intentionally orienting the probe so that the ultrasound beam is normal to the flow axis, a position previously thought to be useless for measuring blood flow.

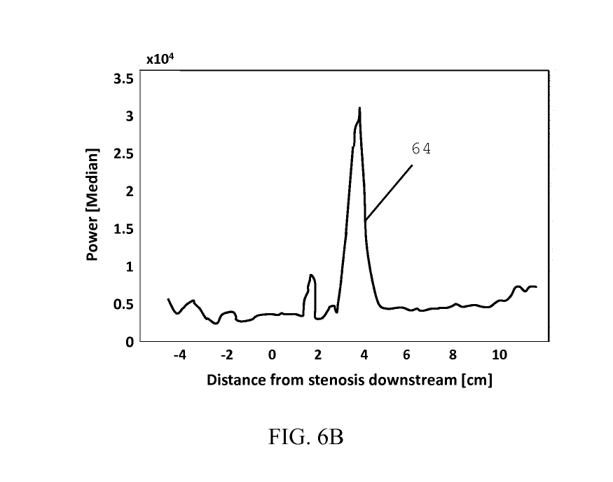

FIGS. 6A and 6B depict actual recordings carried out by means of a probe positioned at an angle of 90° with respect to the flow axis, on a phantom of a coronary artery that has a 1 cm long stenosed segment with a 50% stenosis by diameter (75% stenosis by area). In FIG. 6A, which is plot 62 of (velocity and power) vs. distance, we see the flow velocity along the “artery”, as recorded by a probe positioned at 90° with respect to the flow axis while the probe is moved along the vessel. The 0 point on the x axis is the upstream end of the stenosed segment, and the point marked “a” corresponds to the downstream end of the stenosed segment.

We see that between 1 and 3 cm downstream from the downstream end of the stenosis a symmetric bidirectional increase in flow velocity appears. This represents flow towards and away from the probe, which indicates the presence of turbulence. The turbulence persists for a length of about 2 cm along the axis of flow and has a peak flow velocity (indicated by the arrows b, b′) that occurs about 2 cm from the downstream end of the stenosis. These findings are in agreement with corresponding published reconstructions. See, e.g., S.S. Varghese, S. H. Frankel and P.F. Fischer, Direct numerical simulation of stenotic flows. Part 1. Steady flow, J. Fluid Mech. (2007), vol. 582, pp. 253-280.

Note that while the distance between the downstream end of the stenosis and the center of the turbulent regions was about 2 cm in the above example, it will actually depend on the diameter of the vessel being tested. Typically, the high turbulence will occur at a position that β cm downstream from the downstream end of the stenosis, where β is between about 4-5 times the diameter of the artery that is being imaged.

FIG. 6B shows a plot 64 of the corresponding reflected ultrasound Power, for the same experiment as FIG. 6A. It is clearly seen that the power peaks at the center of the vortex, and it follows that the center of the vortex can be identified by looking for the Power peak. The dimensions of the vortex can also be extracted from the power tracings. Here again, the 0 point on the x axis is the upstream end of the stenosed segment.

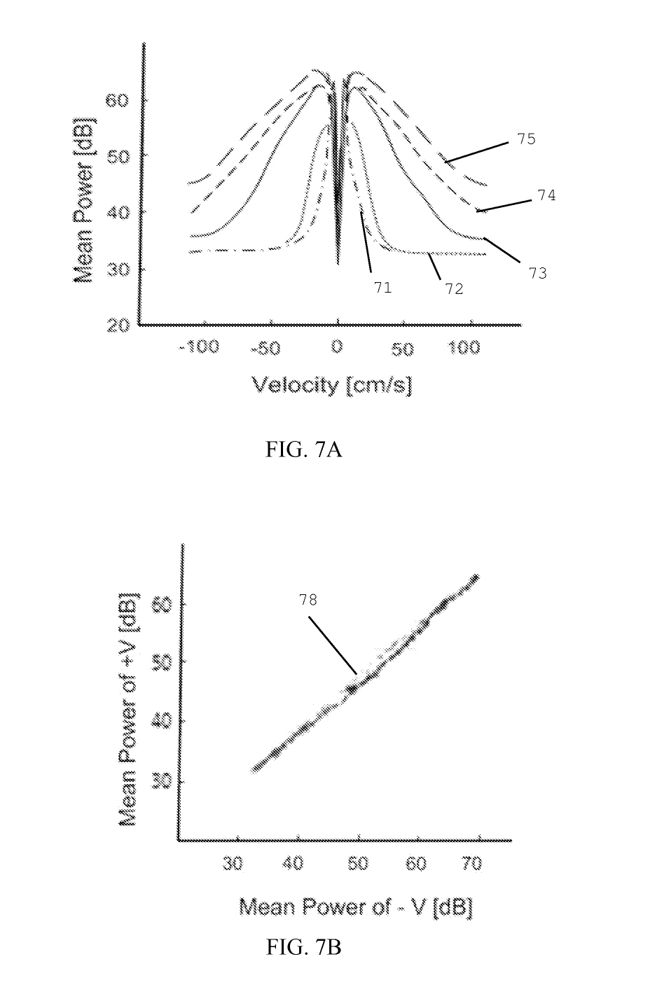

FIG. 7A is a set of Power Spectra recorded by a 2 MHz probe, positioned at an angle of 90° relative to the flow axis, from a phantom representing a coronary artery with two stenoses. One of the stenoses is of 75% by area and the other of 90% by area. Recordings were made during a number of different flow rates in the range of 9.5 to 34 cm/sec. When the probe is positioned at 90°, flows along the vessel (artery) are not recorded so that only the turbulences are registered. The two traces 71, 72 were made at turbulences located about lcm downstream from the 75% stenosis at flow rates of 21 cm/s and 34 cm/s, respectively. The three remaining traces 73, 74, 75 were made at turbulences located about lcm downstream from the 90% stenosis during flows of 9.5, 21 and 34 cm/s, respectively.

The maximal velocities generated by the less severe 75% stenosis correspond approximately to the flow velocity in the unaffected vessel segments. In contrast, the 90% stenosis generates vortex flows having much higher velocities (by a factor larger than 10) and correspondingly higher power as compared with those in the unaffected segments. Note that this highly non-linear behavior can serve to distinguish between low and high grade stenoses. In other words, high power at high velocities is an indication that a severe stenosis may be present upstream.

It therefore makes sense to correlate the presence of a high power level for high velocity components with the presence of a stenosis in the blood vessel. From this correlation, it follows that if the entire blood vessel is tested, and a high power level for high velocity components is not detected, there is probably no severe stenosis in the blood vessel.

Note that the power spectra in FIG. 7A all appear to be symmetric. The level of symmetry can parameterized by determining the correlation between the positive and negative flows as seen for example in FIG. 7B, and this correlation 78 can be used as parametric characterization of the flow and level of turbulence. Since symmetric power spectra are produced when a stenosis is present, especially for power spectra that have high power at high frequency components, the presence of such symmetry can be used to predict or confirm the presence of a stenosis.

Beaming the ultrasound in at an angle that is perpendicular to the direction of blood flow provides the advantage that at this angle all non-turbulent flows in the artery are nulled such that the turbulence is easier to recognize. Accordingly, for best results, the doctor or ultrasound technician who is operating the ultrasound system should manipulate the probe to try to keep the beam as close as possible to perpendicular to the direction of blood flow in the artery. This manipulation may be facilitated by having the operator observe relevant images (e.g., Doppler and/or standard ultrasound images), and will be within the skill level of trained operators. However, even if there probe is not kept perfectly perpendicular, the data will still be usable. It is preferable to keep the deviation from perpendicular below 20°, more preferable to the deviation from perpendicular below 10°, and even more preferable to keep the deviation from perpendicular below 5°.

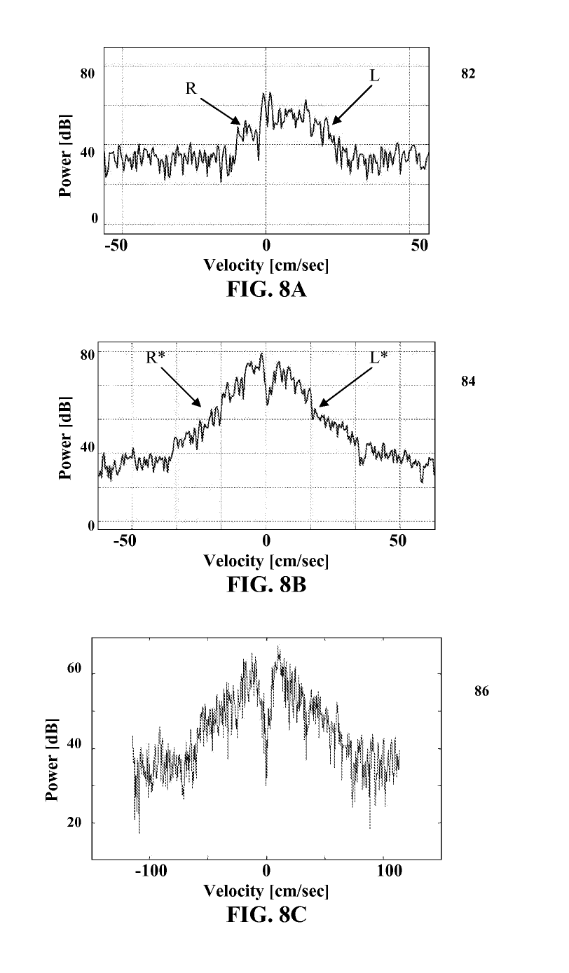

FIGS. 8A-C highlight the differences between the shapes of the power spectra observed in a laminar flow segment and the power spectra observed in a turbulence appearing downstream from a severe stenosis. FIG. 8A depicts a typical power spectrum 82 of blood flow in a normal LAD coronary artery, measured with the us beam at an angle of 80° with respect to the direction of blood flow. The positive and negative parts of the power spectrum, R & L are very different. Such asymmetry is typical of unidirectional normal flow when the ultrasound beam comes in at 80°. FIG. 8B depicts the power spectrum 84 obtained downstream of a stenotic segment (50% stenosis, by diameter) where turbulence occurs, also measured at an angle of 80°. It is seen that the power spectrum becomes highly symmetric, the positive and negative parts of the power spectrum, R* & L* being very similar. FIG. 8C depicts the power spectrum 86 of corresponding turbulence in a phantom, this time measured at an angle of 90°. Note that the spectra 82 and 84 are still usable even though they were captured at a 10° deviation from perpendicular.

III. Multi-Parameter Analysis Together with Perpendicular Data

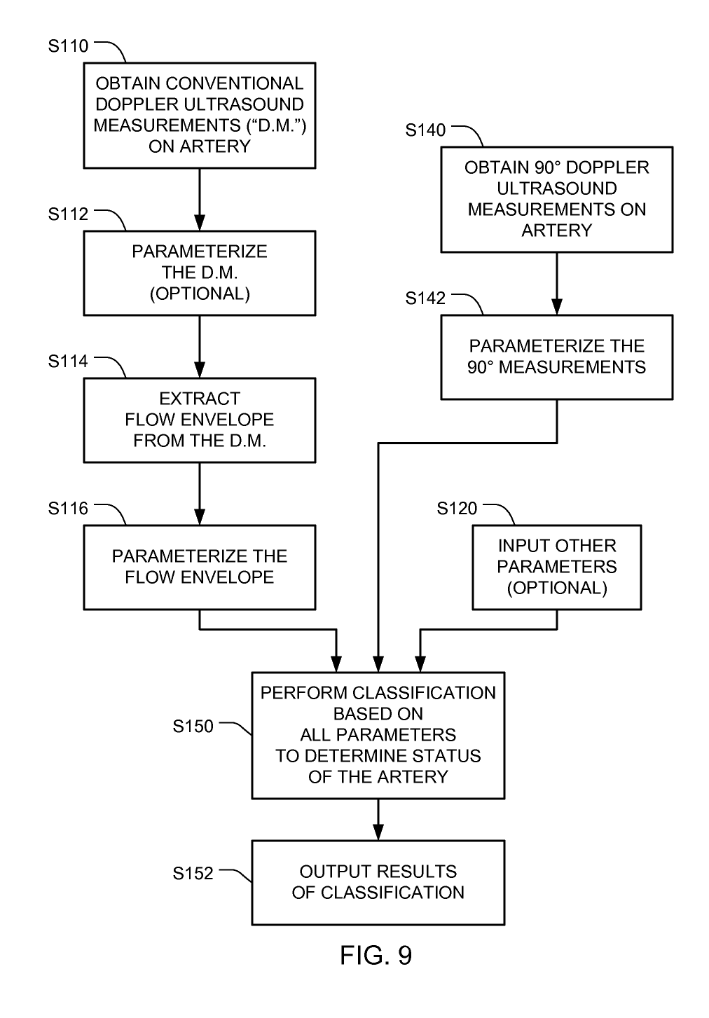

FIG. 9 is a flowchart depicting how the Multi-Parameter approach for detecting a stenosis (described above in section I) can be combined with the Perpendicular Data approach for detecting a stenosis (described above in section II).

In FIG. 9, steps S110-S120 are the same as the corresponding steps described above in connection with FIG. 2. Additional steps S140 and S142 in any time sequence with the other steps S110-S120, or at the same time as those steps. In step S140, Doppler ultrasound measurements are made on the artery (or other vessel) being tested. For best results, the doctor or ultrasound technician who is operating the ultrasound system should manipulate the probe to try to keep the beam as close as possible to perpendicular to the direction of blood flow in the artery, as described above in section II.

After the measurements are obtained, the results are parameterized in step S142, to extract the relevant features from the data. Processing then proceeds at step S150, where classification is done to extract the relevant results from the data. This step is similar to the classification step S132 discussed above in connection with FIG. 2, but the classification model will be different to account for the different inputs. In this embodiment, the classification model preferably includes parameters that are obtained from data that was obtained at or near perpendicular. Examples of suitable parameters would include parameters that reflect high power at high velocities (which are associated with stenoses), and parameters that reflect the level of symmetry between positive and negative velocities (which are also associated with stenoses).

Finally, in step S152, the results of the classification are output in a manner similar to one discussed above for step S132. Note that when the output is made, the output can be configured to indicate the point where the maximum turbulence was detected, or the point where the stenosis is likely to be (i.e., a point downstream from the turbulence).

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof

What is claimed is:

1. A method of detecting a stenosis in a vessel through which a fluid is flowing, the method comprising the steps of:
generating a beam of ultrasound energy;
aiming the beam at a point in the vessel at an angle of less than 20° with respect to a plane that (a) is perpendicular to the direction of flow in the vessel and (b) passes through the point;
using Doppler processing to detect, within the vessel, velocity components of fluid motion that are perpendicular to the direction of fluid flow;
repeating the aiming step and the using Doppler processing step at a plurality of points in the vessel;
identifying a location in the vessel at which the detected velocity components have high power at high velocities; and
determining that there is a high likelihood that a stenosis is present at a position that is upstream from the identified location.

2. The method of claim 1, further comprising the step of outputting an indication of the identified location.

3. The method of claim 1, further comprising the step of outputting an indication that specifies the position that is upstream from the identified location.

4. The method of claim 3, wherein the specified position is between 1 and 3 cm upstream from the identified location.

5. The method of claim 3, wherein the specified position is upstream from the identified location by amount equal to about 4-5 times the diameter of the vessel.

6. The method of claim 1, wherein, in the aiming step, the beam is aimed at an angle of less than 10° with respect to the plane.

7. The method of claim 1, wherein, in the aiming step, the beam is aimed at an angle of less than 5° with respect to the plane.

8. The method of claim 1, wherein the vessel is a blood vessel.

9. A method of detecting a stenosis in a vessel through which a fluid is flowing, the method comprising the steps of:
generating a beam of ultrasound energy;
aiming the beam at a point in the vessel at an angle of less than 20° with respect to a plane that (a) is perpendicular to the direction of flow in the vessel and (b) passes through the point;
using Doppler processing to detect, within the vessel, velocity components of fluid motion that are perpendicular to the direction of fluid flow;
displaying an indication of a power level for the detected velocity components; and
in instances where a high power level for high velocity components is present, correlating the presence of the high power level for high velocity components with the presence of a stenosis in the vessel.

10. The method of claim 9, wherein the step of correlating the presence of the high power level with the presence of a stenosis in the vessel comprises correlating the presence of the high power level for high velocity components detected at a first position in the vessel with the presence of a stenosis in the vessel at second position that is upstream from the first position.

11. The method of claim 9, wherein the step of correlating the presence of the high power level with the presence of a stenosis in the vessel comprises correlating the presence of the high power level for high velocity components detected at a first position in the vessel with the presence of a stenosis in the vessel at second position that is 1-3 cm upstream from the first position.

12. The method of claim 9, wherein the step of correlating the presence of the high power level with the presence of a stenosis in the vessel comprises correlating the presence of the high power level for high velocity components detected at a first position in the vessel with the presence of a stenosis in the vessel at second position upstream from the first position by an amount equal to about 4-5 times the diameter of the vessel.

13. The method of claim 9, wherein, in the aiming step, the beam is aimed at an angle of less than 10° with respect to the plane.

14. The method of claim 9, wherein, in the aiming step, the beam is aimed at an angle of less than 5° with respect to the plane.

15. The method of claim 9, wherein the vessel is a blood vessel.
